(19) United States
(12) Patent Application Publication
(10) Pub. No.: US 2013/0019872 A1
(43) Pub. Date: Jan. 24, 2013

(54) NASOPHARYNGEAL AIRWAY

(71) Applicants: Bahman Guyuron, Lyndhurst, OH (US); Jamie Horvath, Twinsburg, OH (US); Brad A. Pulver, Shaker Heights, OH (US)

(72) Inventors: Bahman Guyuron, Lyndhurst, OH (US); Jamie Horvath, Twinsburg, OH (US); Brad A. Pulver, Shaker Heights, OH (US)

(21) Appl. No.: 13/186,724

(22) Filed: Jul. 20, 2011

(51) Int. Cl. A61M 16/00

(52) U.S. Cl. 128/207.18

(57) ABSTRACT

An anatomically designed egg-shaped or elliptical-shaped nasopharyngeal airway for providing a comfortable and safe air passage through a nose to a nasopharynx includes a cannula having a proximal open end, a distal end, and a sidewall surrounding a lumen of the cannula. The nasopharyngeal airway has a port, which includes two or more openings separated by one or more bridges, in the sidewall, at a distal portion of the cannula. Upper and lower conduits may be located in channels of the sidewall of the cannula for delivery of oxygen and measurement of CO₂ or other purposes.

### TECHNICAL FIELD

The present invention generally relates to an airway adjunct, and more particularly to a nasopharyngeal airway.

### BACKGROUND OF THE INVENTION

A nasopharyngeal airway, also referred to as a nasal trumpet, is a device that is inserted into a patient's nose to maintain an open airway through the nasal passage to the patient's pharynx. The nasopharyngeal airway can reduce or eliminate airway collapse and also may minimize or reduce the possibility that a patient's tongue may slide back in the patient's throat to obstruct the airway. Nasopharyngeal airways commonly are used for patients who are undergoing procedures while under sedation or who are awakening from general anesthesia. Emergency response personnel, such as paramedics, also may use a nasopharyngeal airway for patients who have experienced severe skull or facial trauma, for example, as a result of an accident.

In use, the nasopharyngeal airway is slid through the nostril, along the hard palate (e.g., the bony partition between the oral and nasal cavities) and into the nasopharynx. Inserting and maintaining the nasopharyngeal airway into a patient's nose may cause pain and discomfort for the patient. All conventional nasopharyngeal tubes known to the applicants are round while they are external to the nose, but they become oval or elliptical as they are inserted into the nasal pathway. This deformation occurs as a result of poor design and disregard to the anatomy of the internal nose. However, insertion of such a round tube in a space that is oval or elliptical results in undue pressure on the nasal septum and the turbinates and causes discomfort for the patients requiring more sedation than necessary. If this type of round tube is placed in the nose for a protracted period (several hours) it may even result in a pressure sore on the septum and the turbinates or in a permanent displacement of the turbinates. Further, a distal opening may become blocked by mucous while in a patient's nasopharynx.

Conventional nasopharyngeal airways often consist of a tubular member (also referred to as a cannula) having a circular cross-section. The cannula may have two ends, one for insertion into the patient's nose to the nasopharynx and the other for remaining external to the patient. The cannula may surround a lumen that provides an open pathway to the nasopharynx. Tubes or medical instruments (for example, such as a tube for a carbon dioxide detector, an oxygen supply tube, a suction tube, etc.) can be inserted into the lumen or externally attached to the cannula. When located inside the lumen, these items consume part of the airway and therefore effectively reduce airflow to the nasopharynx. Also, they may reach too far in and curl inside the lumen when fed separately through the lumen. When located external to the cannula, these items may increase the size of the cannula and thus may make the nasopharyngeal airway more uncomfortable for the patient. Furthermore, being unprotected outside the cannula will expose these items to being readily blocked with biological secretions.

### SUMMARY

The present invention provides a nasopharyngeal airway having an oval or egg-shaped contour and a soft, rounded tip to facilitate insertion of the nasopharyngeal airway into the nasal passage, which is also oval in shape at the nostril level and elliptical between the septum and the turbinates, to reduce patient trauma and/or discomfort. The nasopharyngeal airway includes a cannula surrounding a main lumen extending from an open proximal end of the nasopharyngeal airway to the rounded tip. The nasopharyngeal airway also has one or more conduits embedded in the sidewall of the cannula extending from the open proximal end and opening to a portion of the lumen in a distal direction from the proximal end. One conduit (preferably the top conduit) may be larger than the other (preferably bottom) conduit. One conduit (preferably the top conduit) may extend farther into the nasopharyngeal airway than the other (preferably bottom) conduit. The conduits may be inserted into the sidewall or into a channel in the sidewall, or by integrally forming the conduit within the sidewall or a channel therein. By embedding the conduits into the sidewall of the cannula or a channel therein, the conduits do not consume a substantial portion of the lumen. Additionally, complications arising from clogging, tangling, and/or collapsing of the conduits generally can be avoided.

The cross-section of the cannula can be ovular, elliptical, or egg-shaped in order to conform to the shape of the nostril and to further facilitate the insertion of the cannula into the nasal passage and maximize usage of the space in the nasal passage. The nasal passage between the septum and turbinates is not round and the force that converts a round lumen to an oval or egg-shaped lumen causes discomfort in the septum and turbinates. This forceful reshaping of the nasal trumpet can be avoided or reduced by this oval or egg-shaped design of the nasal trumpet and allows the tube to follow the natural contours of the internal nose.

The distal port of the nasal trumpet may be located on the sidewall of the cannula. Further, the port may include two or more openings separated by bridges. The bridges may provide strength or rigidity while the openings may provide for effective and redundant fluid flow of the gasses, (e.g., air and oxygen).

According to one embodiment, the nasopharyngeal airway includes a cannula having a proximal open end, a distal end, and a sidewall surrounding a lumen of the cannula. The cross-section of the cannula is egg-shaped or elliptical. The sidewall may have a port with two or more openings separated by one or more bridges for providing fluid communication between a distal portion of the lumen and the nasopharynx when the cannula is inserted into the nose. The sidewall may have one or more conduits, each having an opening adjacent to the proximal open end of the cannula and an opening in a portion of the cannula in a distal direction from the proximal end for providing fluid communication between the conduit and the lumen. The conduit openings can be elongate in shape so as to reduce or minimize the effects of obstructions, such as mucus or debris.

According to another aspect, the nasopharyngeal airway includes a tubular member having a distal end, a sidewall surrounding a lumen, and a port in the sidewall for providing fluid communication between a distal portion of the lumen and a nasopharynx of a person when the tubular member is inserted into a nose, the port having one or more bridges that divide the port into two or more openings. The nasopharyngeal airway may also include a pair of parallel conduits in the sidewall of the tubular member, each conduit in fluid communication with the lumen through respective conduit openings.

According to another aspect, the nasopharyngeal airway includes a cannula having a proximal open end, a distal end, and a sidewall surrounding a lumen of the cannula. The airway includes a first conduit disposed along an upper side of the cannula and extending from the proximal open end to a first location near the distal end. The airway also includes a second conduit disposed along a lower side of the cannula and extending from the proximal open end to a second location proximal the first location. The upper conduit may have a larger cross-section than the lower conduit. The upper conduit may extend farther than the lower conduit.

According to another aspect, the conduits are fixed in the cannula to prevent longitudinal movement with respect to the cannula thereby preventing kinking of the conduits within the lumen due to overextension.

Herein is provided an anatomically designed egg-shaped or elliptical-shaped nasopharyngeal airway for providing a comfortable and safe air passage through a nose to a nasopharynx including a cannula having a proximal open end, a distal end, and a sidewall surrounding a lumen of the cannula. The nasopharyngeal airway may have a port, which includes two or more openings separated by one or more bridges, in the sidewall, at a distal portion of the cannula. Upper and lower conduits may be located in channels of the sidewall of the cannula for delivery of oxygen and measurement of CO₂ or other purposes.

To the accomplishment of the foregoing and related ends, certain features described hereinafter are particularly pointed out in the claims. The following description and the annexed drawings set forth in detail certain illustrative embodiments. These embodiments, however, are merely indicative of a few of the various ways in which inventive features may be employed.

### BRIEF DESCRIPTION OF THE DRAWINGS

In the accompanying drawings, which are not necessarily drawn to scale:

FIG. 1 is a schematic representation of a nasopharyngeal airway inserted into a patient's nasal passage.

FIG. 2 is an isometric view of an exemplary nasopharyngeal airway.

FIG. 3 is a side view of the nasopharyngeal airway of FIG. 2.

FIG. 4 is a cross-sectional view of the nasopharyngeal airway of FIG. 3 at section lines 4-4.

FIG. 5 is a cross-sectional view of the nasopharyngeal airway of FIG. 3 at section lines 5-5.

FIG. 6 is an isometric view of the back of an exemplary nasopharyngeal airway.

### DETAILED DESCRIPTION

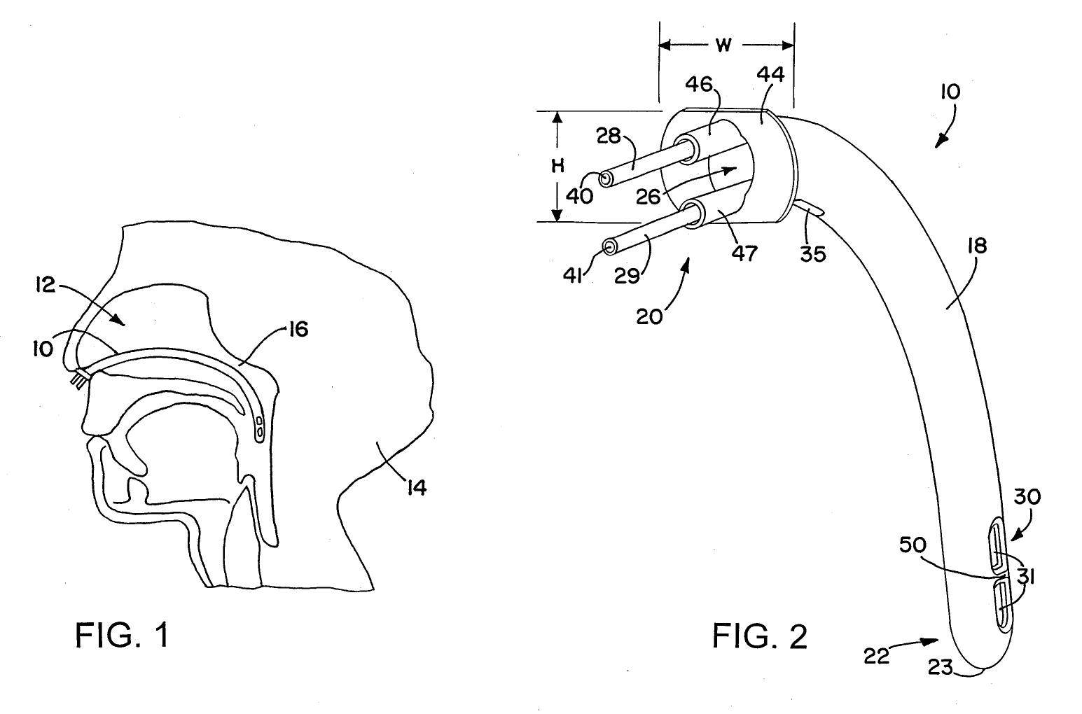

Referring to the drawings, wherein like reference numbers designate like parts in the several figures, and initially to FIG. 1, a schematic representation of a nasopharyngeal airway 10 inserted into a nasal passage 12 of a patient 14 is shown. The nasopharyngeal airway 10 secures an open airway through the patient's nasal passage 12 to the patient's nasopharynx 16.

An exemplary embodiment of the nasopharyngeal airway 10 is shown in FIGS. 2-6. The nasopharyngeal airway 10 may be constructed or formed from a soft, flexible material. The flexible material may facilitate the insertion of the nasopharyngeal airway 10 into the nose and through the nasal passage 12 by allowing the nasopharyngeal airway 10 to bend and flex as may be necessary to navigate through the internal anatomy of the patient's nasal passage 12, for example, the turbinate, the septum, and the hard palate. Exemplary suitable materials include, for example, a biocompatible thermoplastic elastomer, a biocompatible thermoset silicone, or the like. The nasopharyngeal airway can be formed with an injection molding process.

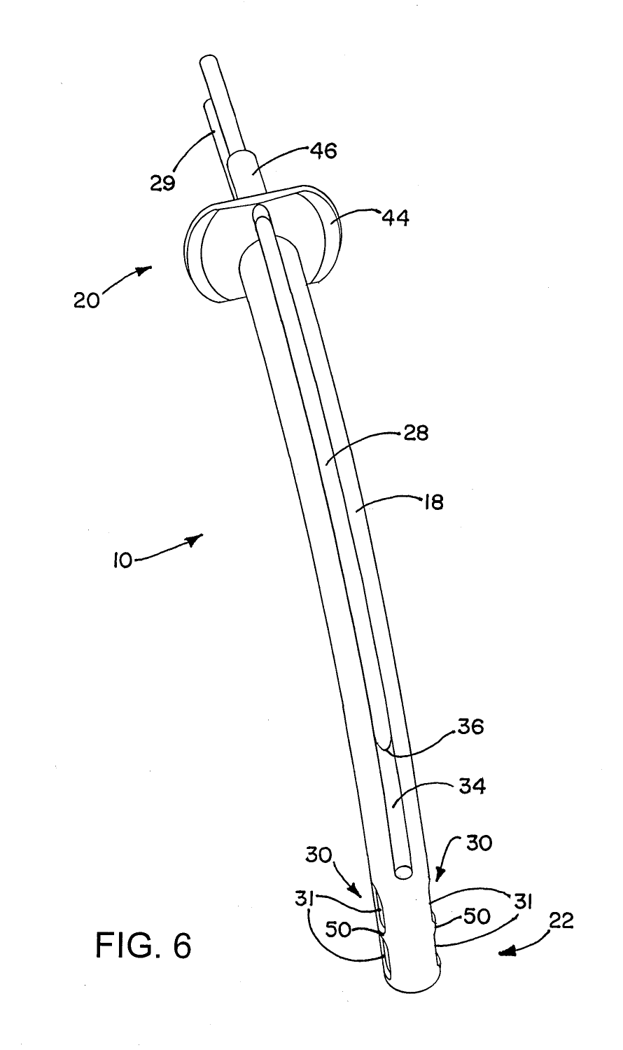

The nasopharyngeal airway 10 includes a cannula 18 having a flared open proximal end 20 and a distal end 22 which may or may not be completely closed and may have a rounded tip 23 for ease of insertion into a patient 14. As shown best in FIG. 6, the outer surface of the cannula 18 has an oval or egg-shaped cross-section. As used herein, “egg-shaped” cross-section means a cross-section having a generally ovular shape having symmetry about its longer or “major” axis, but lacking symmetry about its shorter or “minor” axis. The shape of the outer surface of the cannula 18 has been designed to match (e.g., correspond to) the shape of the nostril opening and the approximate contour of the internal nose governed by the shape of the lower turbinates and the septum. The ovular or egg shape facilitates the insertion of the cannula 18 through the nostril and into the nasal passage 12. By conforming to the general shape of the nostril and the nasal passage 12, pressure applied by the cannula 18 to the turbinate bone and hard palate of the patient can be reduced making the nasopharyngeal airway 10 easier to insert and more comfortable for the patient 14. The cannula 18 also may be formed or molded to have a natural curve so as to conform to the nasal passage and throat of the patient.

The tip 23 of the distal end 22 may be rounded. The tip 23, which is made from the same soft and flexible material as the cannula 18, reduces the likelihood that the distal end of the cannula 18 will catch or grab onto portions of the nasal passage 12 or damage the lining of the septum and turbinates which are very fragile, as the tip 23 is slid through the nasal passage 12 to the nasopharynx 16.

The rounding of the tip 23 also facilitates the insertion of the cannula 18 into the nasal passage 12 by gradually expanding the nasal passage 12 as the cannula 18 is slid into the nostril and over the hard palate. The distal end may also include a taper (not shown) in addition to the rounding to further facilitate insertion comfort. Still further, the cannula 18 may be tapered from the proximal end 20 to the distal end 22. Finally, the distal end 22 may include a hole or opening (not shown) providing fluid communication between the nasopharynx 16 and the lumen 26.

Thus, the rounded tip 23 of the distal end 22 of the cannula 18 facilitates the atraumatic insertion of the cannula 18 into the nasal passage 12. However, because a cannula with an open end may be difficult to insert and may scratch the turbinate and septum of the patient and cause injury and/or bleeding, alternative means of fluid communication may be desirable.

Instead of, or in addition to, an open end, the nasopharyngeal airway 10 may include one or more ports 30 on the distal end 22 of the cannula 18 in a sidewall 24 that surrounds a lumen 26 through the nasopharyngeal airway 10.

The one or more ports 30 may provide fluid communication between the lumen 26 and the nasopharynx 16. In the exemplary embodiment of FIGS. 2-6, and as best seen in FIG. 6, the distal portion of the nasopharyngeal airway has a pair of ports 30 respectively located at opposite positions in the sidewall 24; however, the nasopharyngeal airway may have a different number of ports, for example, only one port or three ports, etc. The nasopharyngeal airway may have a closed distal end 22 in order to minimize the trauma of insertion into a patient 14.

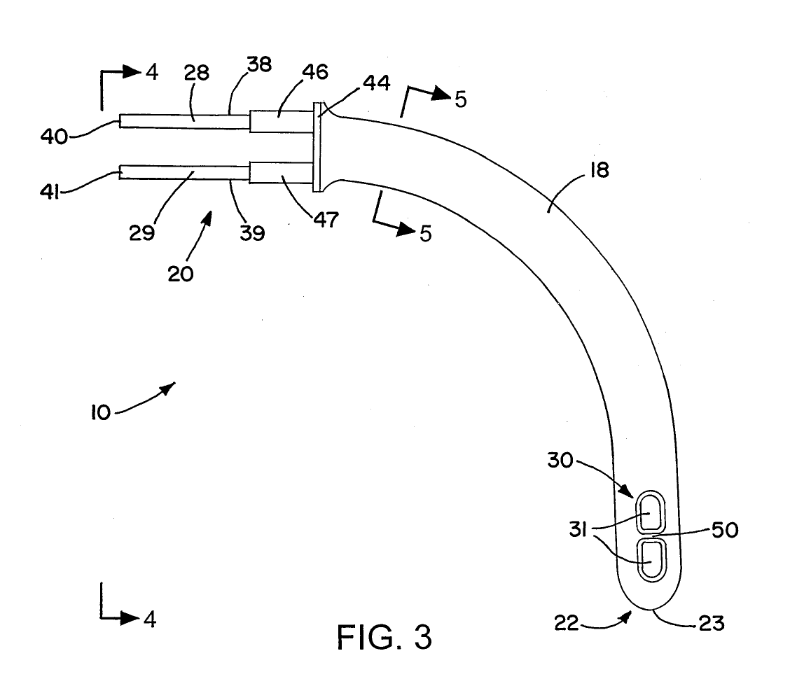

Each port 30 may include two or more openings 31 divided by a bridge 50. The openings may be circular or ovular openings, or may have a different shape, for example, the D-shape shown best in FIG. 3. A port 30 consisting of multiple openings 31 may decrease the possibility of mucous clogging. Further, a large port 30 divided by one or more bridges 50 may increase the strength or rigidity of the cannula 18 near the distal end 22. As used herein, the term “near” may refer in some embodiments to a location closer to one end (e.g., distal) than the other end (e.g., proximal). In other embodiments, the term “near” may indicate a location closer to one end (e.g., distal) than to a center point between the two ends.

The multiple openings 31 in the sidewall 24 provide multiple pathways to the nasopharynx 16. In the event that one of the openings 31 becomes blocked, for example if the distal end 22 of the cannula 18 is pressed against the pharynx or against the tongue, air will still be able to pass through the lumen 26 to the nasopharynx 16 through another opening 31 in the sidewall 24. In such a situation, the lumen 26 is not blocked, but rather remains open to allow for airflow to the nasopharynx 16 through one of the unblocked openings 31.

When slid into the nasal passage 12, the distal end 22 of the cannula 18 may rest on or against the tongue of the patient 14 and/or the pharyngeal tissues in the patient's throat. The openings 31 in the sidewall of the cannula 18 therefore are less likely to be blocked by the tongue or the pharyngeal soft tissues since the distal end 22 is more likely to contact those structures rather than the openings 31 in the sidewall 24. The distal end 22 end protects (e.g., shields) the openings 31 and reduces the potential for blockage of the nasopharyngeal airway from the tongue and/or pharyngeal soft tissues.

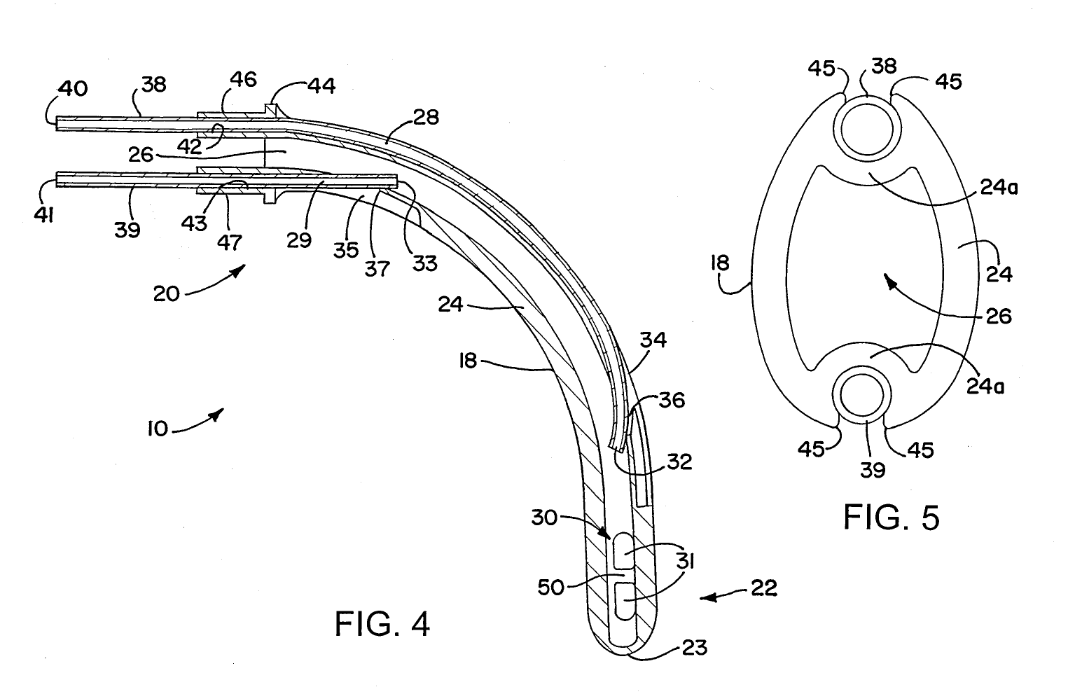

Embedded within channels 34, 35 of the sidewall 24 are one or more conduits 28, 29 having conduit sidewalls 38, 39 defining a flow path that extends along a longitudinal length which extends from proximal openings 40, 41 to distal end openings 32, 33. As shown in FIG. 4, the conduits 28, 29 may extend parallel to one another from the exterior of the cannula 18 into the proximal end of the cannula 18.

The conduits 28, 29 may be disposed anywhere along the sidewall 24 of the cannula 18, but are preferably disposed opposite one another, and are more preferably disposed along the top and bottom of the cannula 18 forming an upper conduit 28 and a lower conduit 29. However, the conduits may, for example, be disposed on the left and right sides of the cannula 18, or may be both located at the top or both located at the bottom of the cannula 18. Alternatively, for example, the conduits may be located at non-orthogonal angles in relation to the cannula 18 cross-section.

The upper conduit 28 may extend the majority of the length of the cannula 18 and open to the main lumen 26 near the distal end 22. The conduits 28, 29 are located in the sidewall 24 of the cannula 18 and are in fluid communication with the lumen 26 via conduit openings 32, 33. The conduits 28, 29 may be fixed within the channels 34, 35 to prevent longitudinal movement with respect to the cannula 18. This movement restriction may prevent the conduits 28, 29 from kinking or catching on something within the main lumen 26, as is sometimes the case with separately-fed conduits.

The upper conduit 28 may have a cross-sectional area that is greater than the lower conduit 29. For example, the upper conduit 28 may have an internal flow path with a cross-sectional area of, preferably between 0.5 mm² and 10.0 mm², and more preferably about 4.4 mm². In contrast, the lower conduit 29 may have a flow path cross-sectional area of, preferably between 0.5 mm² and 10.0 mm², and more preferably about 3.1 mm². In this way, the cross-sectional area of the conduits 28, 29 may be selected, for example, based on different functions. For example, the upper conduit 28, may be of a larger diameter than the lower conduit in order to supply a sufficient amount of oxygen or other gas to a patient 14. In turn, the lower conduit 29 may be, for example, of a smaller diameter, sufficient to deliver exhaled breath to an external CO₂ measuring device (not shown) while taking up as little of the lumen 26 as possible.

Further, the conduits 28, 29 may have openings 40, 41 sized to fit corresponding nipples (not shown) of gas delivery or measuring devices or connected tubing. For example, the openings 40, 41 may fit nipples on disposable tubing used in connection with gas delivery or measuring devices. As another example, the openings 40, 41 may be sized to fit the nipples of permanent tubing used in connection with gas delivery or measuring devices. This latter possibility may, for example, eliminate a need to use separate disposable tubing, and thus lower treatment cost, material waste, and reduce critical set-up or deployment time in emergency situations.

By embedding the conduits 28, 29 in the channels 34, 35 of the sidewall 24 of the cannula 18, the cannula takes advantage of the ovular/egg shape of the nostril and utilizes most or all of the space in the nasal passage while also maximizing the internal width of the lumen 26. In the embodiment of FIGS. 2-6, two conduits 28, 29 are embedded in the channels 34, 35 in the sidewall 24, however, other configurations are possible. For example, the conduits 28, 29 may be integrally formed in the sidewall 24. The conduits can be inserted or otherwise placed in the channels by a secondary assembly process.

As shown in the embodiment illustrated in FIGS. 1-6, conduits 28, 29 are embedded in the sidewall 24 of the cannula 18 by inserting the conduits 28, 29 into respective channels 34, 35 in the sidewall 24. The conduits 28, 29 may be formed from a flexible material so as to facilitate the insertion, removal and/or replacement of the conduits 28, 29 in the channels. Alternatively, the conduits 28, 29 may be formed integrally with or otherwise permanently affixed to the channels 34, 35. The conduits may be made from a biocompatible extruded silicone tubing, for example, or another suitable material. Conduit 28 may have an outer diameter of about 0.5 mm-10.0 mm and preferably about 3.1 mm, and an inner diameter of about 0.2 mm-8.0 mm and preferably about 2.4 mm. Conduit 29 may have an outer diameter of about 0.5 mm-10.0 mm and preferably about 2.7 mm, and an inner diameter of about 0.2 mm-8.0 mm and preferably about 2.0 mm. The conduits 28, 29 can be built as part of the lumen 26 rather than inserting a cannula or tube into the channels.

The channels 34, 35 may extend various lengths along the length of the cannula 18. For example, as shown in FIG. 4, the upper channel 34 may extend along the cannula 18 from the flange 26 to near the distal end 22 of the nasopharyngeal airway 10. Further, the lower channel 35 may extend only partway into the nasopharyngeal airway 10. Regardless of the overall length of the channels 34, 35, the channels 34, 35 may extend beyond the channel openings 36, 37, as shown in FIG. 4, for example, for manufacturing ease.

As shown best in FIG. 4, the flange 44 may have a pair of extensions 46, 47 that surround a portion of the conduits 28, 29 and form a pair of through passages 42, 43. The through passages 42, 43 may provide a passageway from the proximal end 20 of the nasopharyngeal airway 10 to the channels 34, 35, which may extend from a distal side of the flange 44 into the nasopharyngeal airway 10. Each conduit 28, 29 can be inserted into a respective through passage 42, 43 and into the channels 34, 35. Although the illustrated embodiment shows the channels 34, 35 extending to particular locations along the nasopharyngeal airway 10, other configurations are possible. For example, the channels may extend to the tip where the channels 34, 35 may merge with the lumen 26 through channel openings 36, 37 in the sidewall 24 of the cannula 18 or any part of the nasopharyngeal airway 10.

The channels 34, 35 may be shaped to releasably hold the conduits 28, 29. An exemplary embodiment of the profile of the channels 34, 35 is shown in FIG. 5. The channels 34, 35 may have a “C” shape with outer edges 45 that abut against the conduits 28, 29 when the conduits 28, 29 are inserted into the channels 34, 35. The edges 45 may also protect the conduits 28, 29 from being pulled, snagged and/or tangled, etc. while the nasopharyngeal airway 10 is being inserted into the nasal passage 12.

If the conduits are removable, the space between the edges 45 of each respective channel 34, 35 provides a space into which the conduit 28, 29 can be inserted to embed the conduit 28, 29 in the sidewall 24. For example, the space between the edges 45 can be wide enough so as to allow the conduit 28, 29 to be pressed into the channel 34, 35, but narrow enough that the edges 45 limit movement of the conduit 28, 29 in the channel 34, 35 and inhibit easy or unintended removal of the conduit 28, 29 from the channel 34, 35. The conduit 28, 29 and/or the channel 34, 35 may be constructed from a resiliently deflectable material that allows for a temporary deflection and/or deformation during the insertion of the conduit 28, 29 in the channel 34, 35. Upon insertion of the conduit 28, 29 in the channel 34, 35, the structures may naturally return to their original shape, thereby holding the conduit 28, 29 in the channel 34, 35. The conduit 28, 29 can be removed from the channel 34, 35 in a similar manner, for example, by pulling the conduit 28, 29 out of the channel 34, 35 and/or by flexing the channel 34, 35 and/or conduit 28, 29.

It will be appreciated that the edges 45 may include one or more retaining features (not shown) to hold the conduits 28, 29 in the channels 34, 35, or that the conduits 28, 29 may be bonded, welded, or otherwise affixed to the interior of the channels 34, 35. For example, the edges 45 of each channel 34, 35 can be connected to one another at spaced apart locations along the length of the cannula to reinforce the retention of the conduit 28, 29 in the channel 34, 35. Additionally or alternatively, other retaining elements or features can be utilized. The retaining elements may form a generally smooth transition between the edges of the channel 34, 35 and the conduit 28, 29 so as to avoid causing trauma when the nasopharyngeal airway 10 is slid into the nasal passage 12.

Referring back, now, to FIG. 4, the sidewall 24 of the cannula includes a pair of channel openings 36, 37 that provide a pathway from the channels 34, 35 to the lumen 26. Respective distal ends 32, 33 of the conduits 28, 29 can be inserted through the channel openings 36, 37 when the conduits 28, 29 are inserted into the channels 34, 35, thereby providing fluid communication between the conduits 28, 29 and the lumen 26. The channel openings 36, 37 also may be configured to retain the conduits 28, 29 in the channels 34, 35; for example, the conduits 28, 29 can be press-fit into the openings 36, 37 such that the outer wall of the conduits 28, 29 is frictionally retained by the channel openings 36, 37. Alternatively, the conduit 28, 29 can be held by glue or another retaining implement. The connection between the conduit 28, 29 and the opening 36, 37 to the lumen 26 can reduce the likelihood of the conduit 28, 29 from being snagged, tangled or pulled when inserting the nasopharyngeal airway 10 into the nasal passage 12. Additionally, as discussed above, the diameter of the distal ends 32, 33 of the conduits 28, 29 may be greater than the diameter of the conduits 28, 29 at a location proximal to the distal end 32, 33 of the conduit 28, 29 so as to reduce the likelihood of the conduit 28, 29 becoming blocked should debris enter the lumen 26.

Also shown best in FIG. 5, each conduit 28, 29 has a cross-sectional area that is less than the cross-sectional area of the lumen 26. For example, the cross-sectional area of the lumen 26 may be about 5 mm²-about 100 mm² and preferably about 55.5 mm², and the cross-sectional area of the upper conduit 28 may be about 0.5 mm²-about 10.0 mm² and preferably about 4.4 mm² and the cross-sectional area of the lower conduit 29 may be about 0.5 mm²-about 10.0 mm² and preferably about 3.1 mm². The lumen 26 therefore provides a main pathway to the nasopharynx of the patient and the conduits 28, 29 provide secondary or smaller pathways that connect to the distal portion of the lumen 26.

In one embodiment, the upper conduit 28 can be connected to a gas supply (e.g., an oxygen supply) and the lower conduit 29 can be used to measure CO₂ (e.g., by connecting the conduit 29 to a CO₂ sensor), and the gas can be delivered to the distal end portion 22 of the cannula 18 near the ports 30 in the sidewall 24. Delivering the gas to the distal portion of the cannula 18 reduces the likelihood that the gas will accumulate near the proximal end of the cannula 18 where it can be exposed to the external environment (e.g., the operating room or recovery room), which may be undesirable. For example, a spark or other ignition source in the environment could cause oxygen accumulated in a proximal portion of the cannula 18 to ignite. This risk can be reduced by delivering the oxygen to the distal portion of the cannula 18. Additionally, the delivery of oxygen or another gas at a distal portion of the cannula 18 can help to ensure that the patient is receiving the oxygen (via the openings 31 in the cannula 18) and that the oxygen is not being lost to the atmosphere through the open proximal end 20.

As mentioned, one of the conduits 28, 29 may be coupled to a CO₂ sensor for sensing CO₂ to measure respiration. In the illustrated embodiment, the conduit 29 is in communication with the lumen 26 at the proximal end portion 20 of the cannula 18. This location may reduce the likelihood of debris clogging the conduit opening 33, or promote gas mixing within the lumen 26 before being delivered to the measuring device. However, the lower conduit 29 may open to the lumen 26 at any location along the length of the lumen 26. For example, the conduit 28 may open to the lumen 26 at a location closer to the distal end portion 22 of the cannula 18 rather than the proximal end portion 20.

Opening the conduits 28, 29 to the lumen 26 and terminating the conduits 28, 29 within the cannula 18 reduces the likelihood of the conduits 28, 29 being blocked by debris and/or secretions in the nasal cavity because the conduit openings 32, 33 are shielded and protected from direct interaction with the nasal passage 12. Additionally, as the cannula 18 is slid through the nasal passage 12, it is less likely that debris will interfere with the conduit openings 32, 33 because the debris must first pass around the tip 23 of the distal end 22, into one of the openings 31 in the sidewall 24, and then into one of the conduit openings 32, 33. Because the conduit openings 32, 33 are less exposed to the interior of the nasal passage 12, they are less likely to become blocked by debris.

Although not shown, the openings 32, 33 from the conduits 28, 29 to the lumen 26 may be made wider than the cross-sectional area of each conduit 28, 29, by orienting the openings 32, 33 at an oblique angle to the flow path of the conduits 28, 29. For example, the conduits 28, 29 may be cut at a 45 degree angle to enlarge the openings 32, 33 in relation to a fixed cross-sectional area. This opening shape further reduces or minimizes the likelihood that the conduit openings 32, 33 will become blocked by debris. The conduit openings 32, 33 may be elongate in shape, which may further reduce the likelihood that the conduit openings 32 may be blocked by debris.

Referring back to FIG. 2, the proximal end 20 of the cannula 18 may be flared and may form a flange 44. The flange 44 may prevent the nasopharyngeal airway 10 from being inserted too far into the nose by engaging or abutting the sides of the nostrils when the cannula 18 is fully inserted into the nasal passage 12. The flange 44 therefore may prevent the nasopharyngeal airway 10 from being inserted too deep into the patient's head such that it is difficult to remove or to a position where it may harm or otherwise injure the patient.

The flange 44 may be elongate in shape and may, for example, have a width dimension W that is greater than a height dimension H. The flange 44 therefore may be similar in shape to a rectangle having rounded corners, as shown in FIG. 2. A rectangular flange 44 may be less likely than other shaped flanges, for example, a circular shaped flange, to engage or compress the upper lip of the patient when the nasopharyngeal airway 10 is fully inserted into the nose. The rectangular shape also may more comfortable for the patient and less cumbersome for a physician to manipulate. The rectangular flange 44 also may be less likely to interfere with the surgeon's work around the nose during surgery and/or when the patient is under sedation.

Although the drawings and description are directed to one or more embodiments, equivalents and modifications will occur to others skilled in the art upon the reading and understanding of the specification.

Features that are described and/or illustrated with respect to one embodiment may be used in the same way, or in a similar way, in one or more other embodiments and/or in combination with, or instead of, the features of the other embodiments.

What is claimed is:

1. A nasopharyngeal airway for providing an air passage through a nose to a nasopharynx of a patient comprising:
a cannula having a proximal open end, a distal end, and a sidewall surrounding a lumen of the cannula;
a port in the sidewall at a distal portion of the cannula including at least two openings separated by at least one bridge, the openings configured to provide fluid communication between a distal portion of the lumen and the nasopharynx when the cannula is inserted into the patient.

2. The nasopharyngeal airway of claim 1, further comprising a first conduit in the sidewall of the cannula, the first conduit having an opening adjacent to the proximal open end of the cannula and a conduit opening in a distal portion of the cannula for providing fluid communication between the conduit and the distal portion of the lumen.

3. The nasopharyngeal airway of claim 2, further comprising a second conduit in the sidewall of the cannula, the second conduit having an opening adjacent to the proximal open end of the cannula and a conduit opening for providing fluid communication between the second conduit and the proximal end of the lumen.

4. The nasopharyngeal airway of claim 3, wherein the first conduit has a larger cross-sectional area than the second conduit to efficiently deliver oxygen.

5. The nasopharyngeal airway of claim 2, wherein the conduit opening adjacent to the proximal open end of the cannula is sized to fit a corresponding nipple on a permanent connector attached to a gas delivery or measuring device.

6. The nasopharyngeal airway of claim 1, wherein an outer perimeter of the cannula is egg-shaped.

7. The nasopharyngeal airway of claim 1, further comprising a second port in the sidewall at a distal portion of the cannula including at least two openings separated by at least one bridge, the openings configured to provide fluid communication between a distal portion of the lumen and the nasopharynx when the cannula is inserted into the patient

8. The nasopharyngeal airway of claim 1, further comprising a conduit in the sidewall of the cannula, the conduit having an opening adjacent to the proximal open end of the cannula and a conduit opening in a proximal portion of the cannula for providing fluid communication between the conduit and the proximal portion of the lumen.

9. The nasopharyngeal airway of claim 8, wherein the conduit is integral with or affixed to the sidewall of the cannula.

10. The nasopharyngeal airway of claim 8, wherein the conduit is inserted into a channel in the sidewall of the cannula, the channel extending from the proximal open end to a channel opening connecting the channel to the lumen.

11. The nasopharyngeal airway of claim 10, wherein the channel is located on at least one of an upper or lower side of the nasopharyngeal airway.

12. A nasopharyngeal airway for providing an air passage through a nose to a nasopharynx of a patient comprising:
a cannula having a proximal open end, a distal end, and a sidewall surrounding a lumen of the cannula, wherein an outer perimeter of the cannula is egg-shaped.

13. A nasopharyngeal airway for providing an air passage through a nose to a nasopharynx of a patient comprising:
a cannula having a proximal open end, a distal end, and a sidewall surrounding a lumen of the cannula;
a first conduit disposed along a perimeter of the cannula and extending from the proximal open end to a first location near the distal end; and
a second conduit disposed along the perimeter of the cannula and extending from the proximal open end to a second location proximal the first location.

14. The nasopharyngeal airway of claim 13, wherein the second location is near the proximal open end.

15. The nasopharyngeal airway of claim 13, wherein the first conduit has a larger cross-sectional area than the second conduit.

16. The nasopharyngeal airway of claim 13, wherein the conduits are disposed in channels formed on the outside of the cannula, and the conduits enter the lumen through channel openings.

17. The nasopharyngeal airway of claim 16, wherein the channels extend past the channel openings.

18. The nasopharyngeal airway of claim 13, wherein the outside of the cannula is egg-shaped.

19. The nasopharyngeal airway of claim 13, wherein conduit openings adjacent to the proximal open end of the cannula are sized to fit corresponding nipples on permanent connectors attached to one or more gas delivery or measuring devices.

20. The nasopharyngeal airway of claim 13, wherein the first conduit is disposed along a top of the cannula and the second conduit is disposed along a bottom of the cannula.

21. The nasopharyngeal airway of claim 13 wherein the conduits are fixed in the cannula to prevent longitudinal movement with respect to the cannula thereby preventing kinking of the conduits within the lumen due to overextension.
